(19) United States
(12) Patent Application Publication
(10) Pub. No.: US 2015/0177236 A1
(43) Pub. Date: Jun. 25, 2015

(54) COMBINED CHEMILUMINESCENCE AND ELISA AUTOMATED SAMPLE READER

(71) Applicant: Gold Standard Diagnostics, Davis, CA (US)

(72) Inventors: Peter Van Praet, Haasrode (BE); John Griffiths, Davis, CA (US); Jennifer Roth, Sacramento, CA (US); Karl Benjamin, Davis, CA (US)

(21) Appl. No.: 14/211,705

(22) Filed: Mar. 14, 2014

Related U.S. Application Data
(60) Provisional application No. 61/790,737, filed on Mar. 15, 2013.
(60) Provisional application No. 61/847,459, filed on Jul. 17, 2013.

(51) Int. Cl. G01N 33/543

(52) U.S. Cl. CPC G01N 33/54386

(57) ABSTRACT

The automated reader operates within an enclosure also containing a sample rack and reagent rack, as well as a microtiter plate with multiple wells therein. The machine automatically loads sample and reagent into a well of the microtiter plate according to a testing protocol. The specimen can then be moved along with the microtiter plate into a slot of the reader and aligned with either a light source and detector for an optical density read or aligned with a photon counter for a chemiluminescence read. The reader includes a shroud assembly which can be raised and lowered by a lift assembly to occlude a read point from photons other than those emanating from the sample.

### CROSS-REFERENCE TO RELATED APPLICATIONS

This application claims benefit under Title 35, United States Code §119(e) of U.S. Provisional Application No. 61/790,737 filed on Mar. 15, 2013 and U.S. Provisional Application No. 61/847,459 filed on Jul. 17, 2013.

### FIELD OF THE INVENTION

The following invention relates to automated biological sample testing equipment which can perform tests such as ELISA tests and CLIA tests. More particularly, this invention relates to robotic analyzers which can read both optical density of a sample and chemiluminescence of a sample in a single machine.

### BACKGROUND OF THE INVENTION

One form of medical diagnostic test is referred to as ELISA which stands for “Enzyme Linked Immuno Sorbent Assay.” The test can be used to determine whether exposure has occurred to an infectious agent which has caused antibodies to be created. In an ELISA test a sample of blood is added to proteins from the infectious agent. Any antibodies in the blood that combine with the proteins, indicating a history of infection, are detected by adding a test antibody linked to an enzyme that causes a color change.

To efficiently perform ELISA tests, it is known to utilize a robotic analyzer, also referred to as a “reader.” Examples of such robotic analyzers are included in U.S. Published Patent Application Nos. 2012/0178170 and 2012/0182556, each incorporated herein by reference.

In some instances, it is desirable to perform multiple different forms of immuno assays on a sample. In such instances, a larger sample must be collected and then split for the separate immuno assays to be performed. Even if the assay performing equipment is in the form of a robotic analyzer which has automated the performance of the immuno assay, performing two immuno assays requires twice the sample size and either twice the time or twice the amount of equipment (or both). Many immuno assays have similar steps and functions included therein. Thus, it is inefficient to have two separate robotic analyzer readers with many similar components each being utilized for only one type of immuno assay.

### SUMMARY OF THE INVENTION

With this invention a robotic analyzer is provided which can provide both an ELISA immuno assay test and also perform a chemiluminescence immuno assay test (also referred to as CLIA). Such a combo reader can utilize a common housing, common computer interface, and common robotics for moving samples around within the analyzer. Elements which perform the various specific functions of the particular immuno assay being conducted are aggregated within the analyzer and the appropriate equipment is utilized in the appropriate sequence to perform each of the tests, including ELISA and CLIA. A single sample can have both ELISA and CLIA immuno assays performed in this manner, both rapidly and efficiently.

The particular equipment utilized in performing the CLIA immuno assay test can be any of a variety of known sets of equipment and testing procedures known in the prior art for performing the CLIA test. Generally, reacting agents are added to the sample to be tested. Other required steps can also be performed to prepare the sample for “reading,” including placing the sample into a well of a microtiter plate within the robotic analyzer. Finally, some form of measurement is performed to measure the chemiluminescence which results. In one embodiment this measurement is in the form of a photon counter which can count the relative chemiluminescence resulting from performing of the CLIA test, which data from the photon counter can then be read and interpreted according to the CLIA test protocol.

Preferably, the reader that is used to measure chemiluminescence can also be used to measure optical density. In particular, the reader also includes a light source and a detector with the microtiter plate locatable with a sample well between the light source and the detector to take a measurement correlatable with optical density.

### OBJECTS OF THE INVENTION

Accordingly, a primary object of the present invention is to provide an automated reader for reading both optical density and chemiluminescence, such as in the performance of ELISA tests or CLIA tests.

Another object of the present invention is to provide a reader which utilizes a common body for both optical density and chemiluminescence sensing of samples within wells of a microtiter plate adjacent the reader body.

Another object of the present invention is to provide a method for performing both ELISA tests and CLIA tests with a common read body within an automated sample analyzer.

Another object of the present invention is to provide a chemiluminescence detection system which accurately measures chemiluminescence emitted from a sample.

Another object of the present invention is to provide a machine which automates the performance of both optical density tests and chemiluminescence tests in a reliable and efficient manner

Another object of the present invention is to provide a machine which can perform both optical density and chemiluminescence tests with common robotic motion elements for moving a single reader body and a single microtiter plate relative to each other.

Another object of the present invention is to provide a platform including data storage and management software for converting signals received from optical density sensors and chemiluminescence sensors into data correlating with optical density and chemiluminescence of a sample.

Other further objects of the present invention will become apparent from a careful reading of the included drawing figures, the claims and detailed description of the invention.

### BRIEF DESCRIPTION OF THE DRAWINGS

FIG. 1 is a perspective view of a machine having various equipment therein including a reader for performance of ELISA and CLIA tests according to this invention and other tests that require reading of optical density or chemiluminescence of samples handled within the machine.

FIG. 2 is a perspective view of the reader of this invention with a microtiter plate located within a slot of the reader for conducting of optical density or chemiluminescence tests.

FIG. 3 is a perspective view of the reader of this invention with portions of an outer housing removed to reveal interior details.

FIG. 4 is a sectional perspective view similar to that which is shown in FIG. 3 and further illustrating internal structures of the reader according to this invention.

FIG. 5 is a detail of a portion of that which is shown in FIG. 4.

FIG. 6 is an exploded parts view of that which is shown in FIG. 5.

FIG. 7 is an exploded parts view of a motor located within the reader housing and which adjusts an elevation of a shroud assembly through a lift assembly.

FIG. 8 is a perspective view of an interconnection between a photon counter and a fiberoptic line within the reader of this invention.

### DESCRIPTION OF THE PREFERRED EMBODIMENT

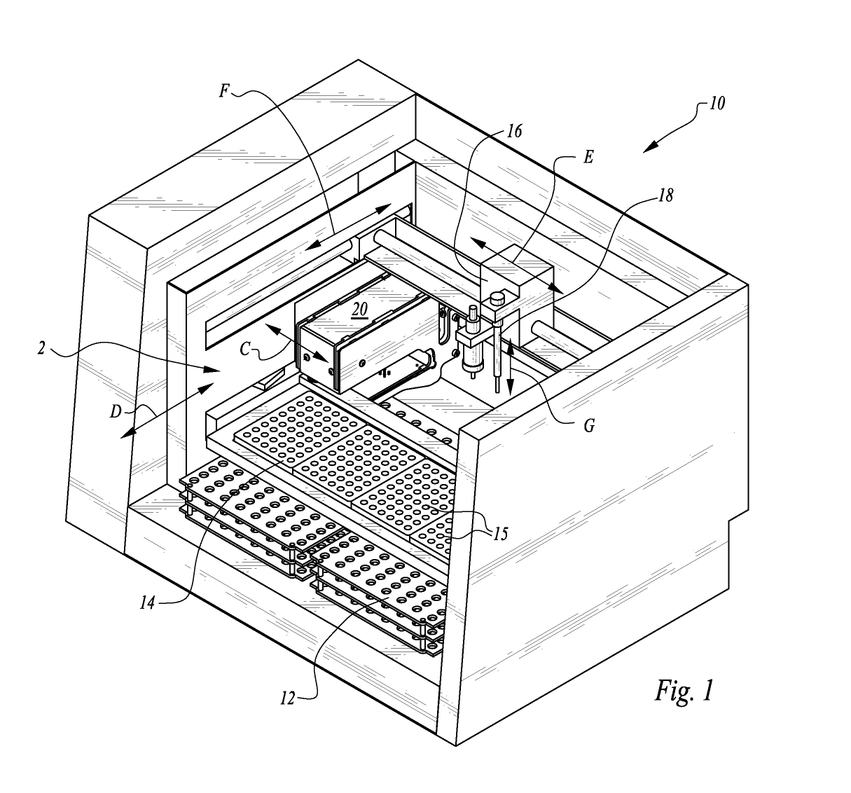

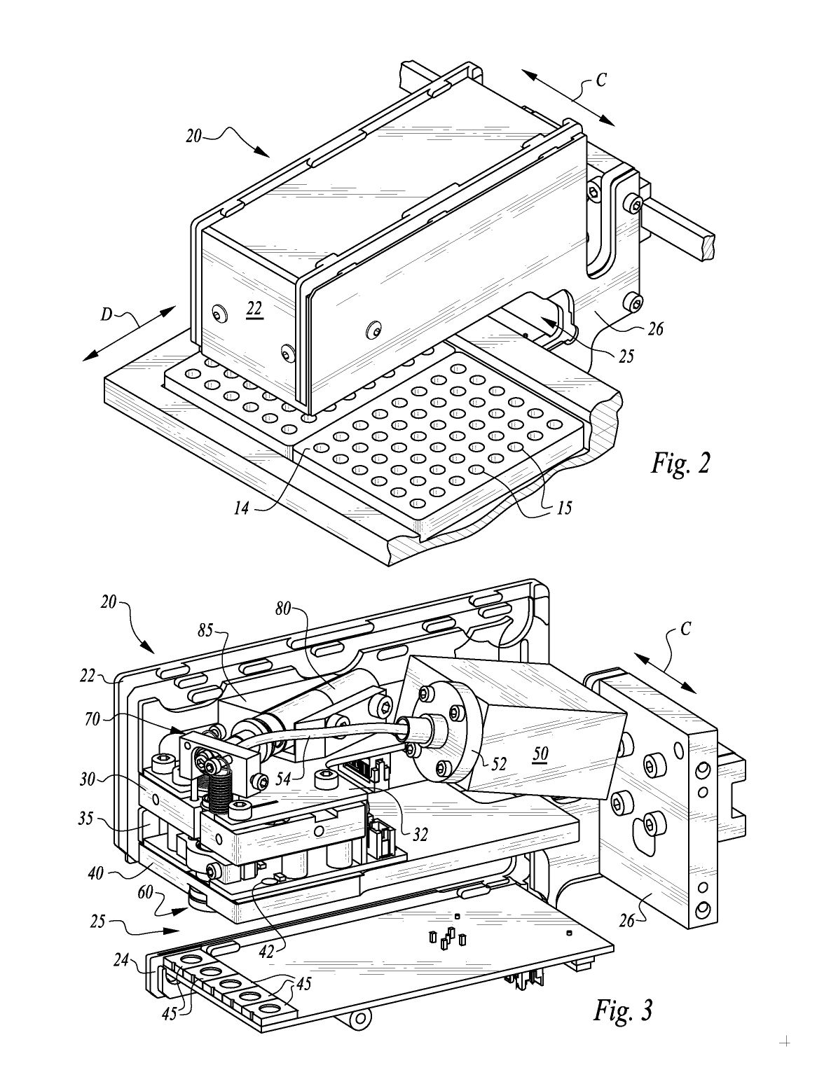

Referring to the drawings, wherein like reference numerals represent like parts throughout the various drawing figures, reference numeral 10 is directed to a machine for performance of both optical density and chemiluminescence tests, such as optical density tests in performance of an ELISA test or chemiluminescence tests in performance of a CLIA test (FIG. 1). The machine is configured to allow samples to be easily loaded thereinto and then to automate the performance of particular test protocols in a highly reliable and automated fashion. A reader 20 (FIGS. 1 and 2) can then be utilized to read the sample in terms of optical density (such as for an ELISA test) or chemiluminescence (such as for a CLIA test).

In essence, and with particular reference to FIGS. 1 and 2, basic details of the machine 10 and general details of the reader 20 are described, according to this preferred embodiment. The machine 10 is preferably a generally orthorhombic structure with an undersurface parallel and spaced from a top surface and with substantially vertical side walls parallel and spaced from each other and a substantially vertical front and rear wall parallel and spaced from each other. The machine 10 can thus set upon a horizontal surface such as a counter. An enclosure 2 is located within an interior of the machine 10 with a front generally being accessible into the enclosure, as well as optionally also a top.

The enclosure 2 includes various elements which are utilized for loading and storing of samples, loading and storing of reagents, and motion of various structures including a microtiter plate 14, a reader 20 and a microsyringe 18 or other fluid handling device for aspirating and dispensing fluids from various different locations within the enclosure 2. A camera can also be provided such as adjacent the microsyringe 18 for recording the operation of the machine 10 and for providing photographic images as another form of read of a sample after a test has been performed. Structures within the enclosure include a sample rack 12 preferably at a lower level which includes a series of locations where samples can be placed, such as within test tubes. Reagent wells are provided within a reagent rack which is typically in a rear right corner of a lower level of the enclosure 2.

The microtiter plate 14 is provided at a midlevel within the enclosure 2. This microtiter plate 14 is a generally planar rigid structure oriented substantially horizontally and including a plurality of wells 15 extending into an upper surface thereof. The microtiter plate 14 is preferably formed of a transparent or at least partially translucent material so that optical density reads can be performed through the microtiter plate 14 with a light source on one side of the microtiter plate 14 and with a detector on an opposite side of the microtiter plate 14. The microtiter plate 14 preferably rests upon a platform which is configured to slide forwardly and rearwardly within a horizontal plane within the enclosure 2 (along arrow D of FIGS. 1 and 2).

An upper carriage 16 is provided as an upper level within the enclosure 2. This upper carriage 16 preferably includes a bar which laterally spans the enclosure 2 which bar can move front to rear (along arrow F of FIG. 1) and with a carriage 16 which can move laterally along the rail which carries the microsyringe 18 and optionally also a camera. This upper carriage 16 moves laterally (along arrow E of FIG. 1) so that, along with movement of the bar, the microsyringe 18 can be moved front to back and laterally within the enclosure 2, so that the microsyringe 18 can access each of the locations within the sample rack 12 and also reach each of the locations of the wells 15 within the microtiter plate 14, and also the various different containers within the reagent rack.

Appropriate robotics including motors and interconnecting links control motion of the microtiter plate 14 (along arrow D) and motion of the microsyringe 18 through the upper carriage 16 (along arrows E and F). Similarly, the reader 20 is configured to move laterally (along arrow C of FIGS. 1 and 2).

The reader 20 is generally configured with an upper housing 22 above a lower housing 24 and with a slot 25 therebetween. A yoke 26 at a rear side of the reader 20 supports the upper housing 22 and lower housing 24 and provides for lateral motion of the reader 20 (along arrow C). The slot 25 has a depth sufficient so that each of the wells 15 within the microtiter plate 14 can be brought into alignment with structures within the reader 20 and near a front tip thereof which measure optical density or which measure chemiluminescence.

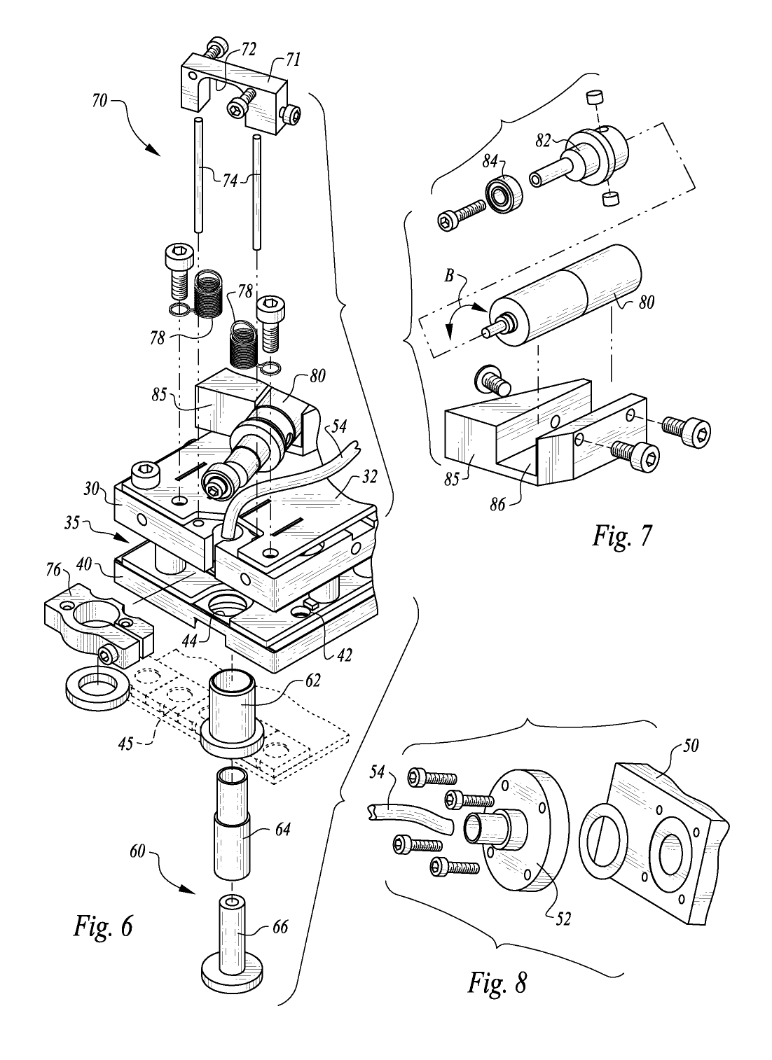

With particular reference to FIGS. 3-6, particular details of the structures which read optical density and structures which read chemiluminescence within the reader 20 are described, according to this preferred embodiment. Within the reader 20, a shelf 30 is provided horizontally, generally at a midpoint within the upper housing 22. This shelf 30 preferably is located just below a light source 32 and is spaced above a floor 40 within the upper housing 22 by a gap 35 beneath the shelf 30 and above the floor 40. Light holes 42 pass through the shelf 30 and also through the floor 40. The light source 32 is aligned with these light holes 42 so that light can shine down through the light holes 42.

The light source 32 is preferably in the form of a printed circuit board with LEDs mounted thereon. A separate LED can be provided for each of the light holes 42. In the embodiment shown, five light holes 42 are provided. As an alternative, a single LED could be provided with fiberoptic cables routing the LED light to each of the light holes 42. Detectors 45 are located in the lower housing 24 beneath each of the light holes 42. These detectors 45 are each aligned with one of the light holes 42 so that they can detect an amount of light from the light source 32 which makes its way through a specimen within one of the wells 15 of the microtiter plate 14. The amount of light passing through the specimen within the well 15 correlates with the optical density of the sample. The more optically dense the sample is, the lesser amount of light passes from the light source 32 through the light hole 42 down to the detectors 45.

Preferably, each of the detectors 45 detects a different wavelength of light. This can be done by having the detectors 45 optimized for detecting different frequencies of light or filters can be utilized so that only light frequencies of particular ranges can pass along the pathway from the light source 32 through the light holes 42 and down to the detectors 45. As another alternative, LEDs having a limited wavelength of light can be utilized so that only particular frequencies of light are provided by the light source 32.

Because the detectors 45 are beneath each of the light holes 42 and beneath the light source 32, a well 15 can have a sample therein read by the reader 20 by simultaneously aligning the microtiter plate 14 and the reader 20 to align the appropriate light hole 42 and detector 45 with the well 15 to have its sample detected. The measurement that is detected can then be correlated with the particular sample which was originally taken from one of the locations in the sample rack 12 and automatically correlated with other information relating to the sample. This data can be stored and further processed into a meaningful test result, according to the particular assay protocol involved.

To facilitate reading of chemiluminescence as well as optical density, an additional bore 44 passes through the floor 40. A photon counter 50 is located within the upper housing 22 of the reader 20 and a fiberoptic line 54 passes from a fiber coupling assembly 52 on the photon counter 50 down through the bore 44 at a read point for reading of chemiluminescence. This bore 44 is preferably slightly closer to a distal end of the reader 20 than the light holes 42. Similar robotics are utilized to align this read point beneath the bore 44 with one of the wells 15 on the microtiter plate 14, preferably by movement of the microtiter plate 14 and/or the reader 20 so that chemiluminescence emanating from a sample within the well 15 of the microtiter plate 14 can be sensed by the photon counter 50 through the fiberoptic line 54.

Preferably, the enclosure 2 of the machine 10 is configured with a hood which can occlude all light within the machine 10 so that only photons emanating from the sample in the form of chemiluminescence can be detected by the photon counter 50. Furthermore, the light source 32 (and any other lights) within the reader 20 and/or enclosure 2 can be turned off during operation of the photon counter 50 to avoid any counting of photons emanating from the light source 32.

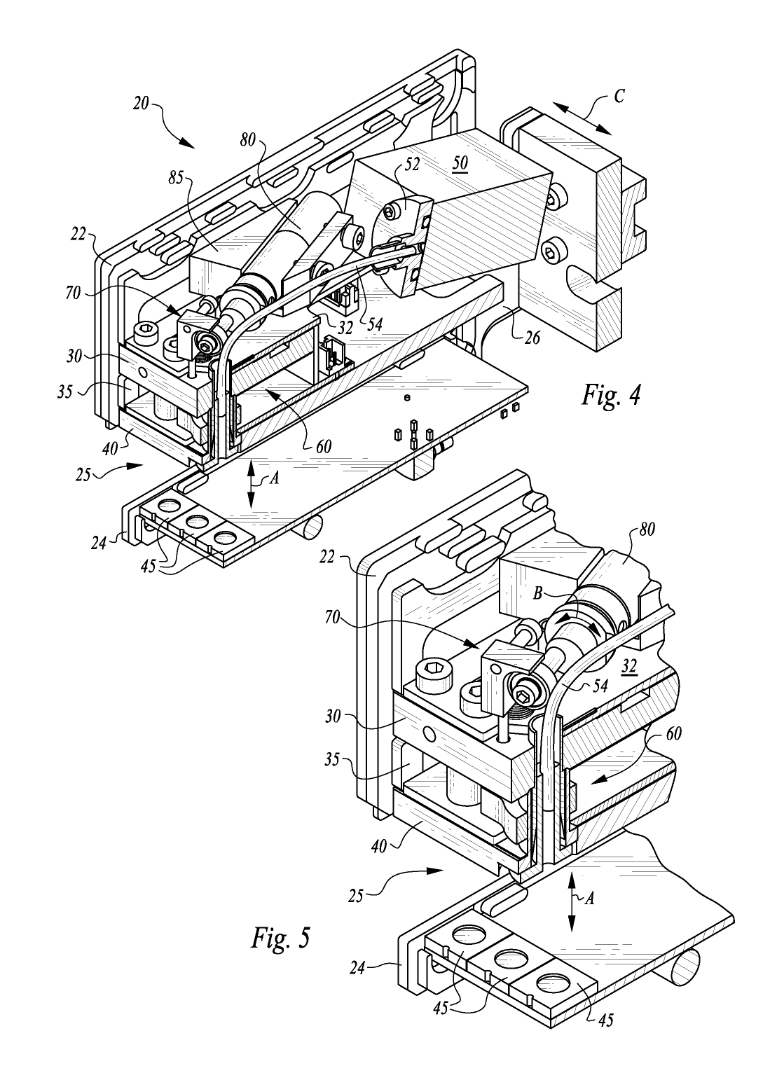

With particular reference to FIGS. 3-8, particular details of a shroud assembly 60 and lift assembly 70 for further occluding any photons other than those emanating from the sample from being detected by the photon counter 50, are described according to this preferred embodiment. The shroud assembly 60 (FIGS. 4-6) includes three separate structures in this embodiment including a hat 62, holder 64 and stop 66. In other embodiments, a single or other numbers of separate structures could comprise an alternative shroud assembly. With this shroud assembly 60, at least one of the structures is in the form of a hat 62 which has an elongate cylindrical portion and a generally flat annular portion located at a lower end of the elongate cylindrical portion. The elongate cylindrical portion has a hollow interior which is aligned with the fiberoptic line 54 and with the fiberoptic line extending down into the hat 62 at least partially. In this embodiment, the holder 64 is attached to the fiberoptic line 54 and the stop 66 keeps the holder 64 and fiberoptic line 54 from becoming displaced vertically totally out of the bore 44 at the read point.

The shroud assembly 60 blocks any photons which might come into the read point laterally between an end of the fiberoptic line 54 and the floor 40 and slightly below the floor 40 of the reader 20. In this way, substantially only photons emanating from a specimen within the well 15 of the microtiter plate 14 can shine up into the shroud assembly 60, through the fiberoptic line 54 and to the photon counter 50 for counting. As an alternative to the fiberoptic line 54, the light path to the photon counter 50 could be direct line of sight or could use mirrors to direct light to the photon counter 50.

The shroud assembly 60 can be dropped down during the instant of reading to further preclude counting of photons coming from sources other than the specimen. Further steps could additionally be taken if desired including utilizing a microtiter plate 14 formed of an opaque material and allowing the shroud assembly 60 to come down into contact with the microtiter plate 14 surrounding the well 15 during reading with the photon counter 50. As another alternative, a sample that is known to be non-chemiluminescent or an empty well 15 of a microtiter plate 14 can be first utilized to calibrate the machine 10 so that a particular amount of photons counted by the photon counter 50 can be considered to be merely background photonic radiation to be subtracted from any actual read by the photon counter 50, so that remaining photons would be those to be counted as resulting from chemiluminescence of a specimen.

To allow for raising and lowering of the shroud assembly 60, a lift assembly 70 is preferably provided. In this embodiment, the lift assembly 70 includes a bridge 71 above the shelf 30. The bridge 71 includes an arch 72 on an undersurface thereof and pillars 74 which extend down from lateral edges of the bridge 71 down to a clamp 76. The clamp 76 is configured to attach to some portion of the shroud assembly 60, such as the hat 62. The pillars 74 extend through the shelf 30 so that the clamp 76 is located in the gap 35 between the shelf 30 and the floor 40 and the bridge 71 is located above the shelf 30.

Springs 78 are coupled to the bridge 71 and to the shelf 30 and bias the lift assembly toward a lowered position. In alternative embodiments, springs or other structures could be provided to bias the bridge 71 in an elevated position.

To move the lift assembly 70 up and work against the springs 78, a motor 80 is utilized. In particular, the motor 80 has a rotating output shaft which is coupled to an eccentric fitting 82. A movable roller tip 84 is coupled to the eccentric fitting 82 along a line offset with a centerline of an output shaft of the motor 80. Thus, when the motor 80 rotates, the roller tip 84 moves up and down slightly. The roller tip 84 is configured to roll against the arch 72 on an underside of the bridge 71, so that when the motor 80 rotates (such as a quarter turn) the movable roller tip 84 acts against the arch 72 to raise the bridge 71, along with raising the clamp 76 and in turn raising the hat 62 or other portion of the shroud assembly 60.

This motor 80 is preferably mounted within a bracket 85 (FIG. 7) which includes a trough 86 therein into which the motor 80 can reside. Bolts are extended laterally from the bridge 71 to allow for attachment of upper ends of the springs 78 thereto. Bolts are also utilized to secure the motor 80 within the trough 86 of the bracket 85 so that the motor 80 resists motion.

While the photon counter 80 is shown located within the upper housing 22, it is conceivable that the photon counter 50 could be configured to read a sample within a well 15 from below rather than from above, through a transparent or translucent microtiter plate 14. However, it is generally preferable that the photon counter 50 read photons emanating from the sample from above to allow for direct sampling and to utilize the space generally available within the larger upper housing 22 of the reader 20.

This disclosure is provided to reveal a preferred embodiment of the invention and a best mode for practicing the invention. Having thus described the invention in this way, it should be apparent that various different modifications can be made to the preferred embodiment without departing from the scope and spirit of this invention disclosure. When structures are identified as a means to perform a function, the identification is intended to include all structures which can perform the function specified. When structures of this invention are identified as being coupled together, such language should be interpreted broadly to include the structures being coupled directly together or coupled together through intervening structures. Such coupling could be permanent or temporary and either in a rigid fashion or in a fashion which allows pivoting, sliding or other relative motion while still providing some form of attachment, unless specifically restricted.

What is claimed is:

1. A combined chemiluminescence and ELISA automated sample reader, comprising in combination:
a reader body having an upper housing and a lower housing spaced apart by a slot;
a microtiter plate sized to fit within said slot, said microtiter plate having a plurality of wells on an upper surface thereof;
at least one light source supported by said reader body, said light source coupled to a power supply and oriented to shine light across said slot;
at least one light detector on a side of said slot opposite said light source;
said microtiter plate and said reader body movable relative to each other to align a plurality of said wells individually along a path between said light source and said light detector; and
at least one of said upper housing and said lower housing having a photon counter oriented to detect photons at a read point of said reader body, when photons are emitted from a sample located in one of said wells of said microtiter plate.

2. The reader of claim 1 wherein a fiberoptic line extends from said photon counter to adjacent said read point, said reader body and said microtiter plate movable to align said read point with a plurality of said wells in said microtiter plate individually, for detection of photons emitted from a sample located within one of said wells by passage of the photons through said fiberoptic line to said photon counter.

3. The reader of claim 2 wherein a shroud assembly is located adjacent said read point, said shroud assembly occluding light from accessing said photon counter through said fiberoptic line from sources other than the sample within one of said plurality of wells of said microtiter plate.

4. The reader of claim 3 wherein said shroud assembly includes a hat structure having an elongate cylindrical portion and an annular portion at an end thereof below said elongate cylindrical portion, said elongate cylindrical portion generally aligned with a central axis of said fiberoptic line, said annular portion of said hat extending below a lower surface of said upper housing of said reader body.

5. The reader of claim 4 wherein said hat is coupled to a lift assembly configured to adjust a vertical position of said hat, said lift assembly including a clamp secured at least indirectly to said hat, a bridge coupled to said clamp with an arch on an underside of said bridge, and with a movable tip coupled to an eccentric fitting rotatably mounted upon a motor such that when the motor rotates the movable tip is caused to be elevated and lowered while abutting said arch of said bridge, such that said motor rotation causes said bridge to be raised and lowered, and in turn said hat is caused to be raised and lowered.

6. The reader of claim 1 wherein said light source includes at least one LED located within said upper housing, and wherein said read point of said photon counter is also located within said upper housing.

7. The reader of claim 6 wherein said upper housing includes a plurality of light holes which are each configured to allow LED light to pass downward therethrough and toward said lower housing, said lower housing including a plurality of light detectors thereon, each of said light detectors aligned with one of said plurality of light holes; and
wherein at least one bore extends down from said upper housing at said read point with said fiberoptic line extending from said photon counter to said read point and extending at least partially down through said bore with said fiberoptic line pointing toward said read point within said slot between said upper housing and said lower housing.

8. The reader of claim 1 wherein said reader body is configured to be movable laterally, and wherein said microtiter plate is configured to be movable within a horizontal plane into and out of said slot of said reader body and in a direction substantially perpendicular to motion of said reader body.

9. The reader of claim 8 wherein said read point is alignable with individual wells of said microtiter plate and said light source is alignable with individual wells of said microtiter plate through a combination of motion of said reader body and motion of said microtiter plate.

10. The reader of claim 9 wherein a shroud assembly is located adjacent said read point to occlude light from sources other than one of said wells of said microtiter plate, and wherein said shroud assembly is coupled to a lift assembly, said lift assembly adapted to raise and lower said shroud assembly toward and away from said microtiter plate adjacent said read point.
